(19) United States
(12) Patent Application Publication
(10) Pub. No.: US 2008/0161754 A1
(43) Pub. Date: Jul. 3, 2008

(54) METHOD AND APPARATUS FOR INFUSING LIQUID TO A BODY

(75) Inventor: April A. Marano-Ford, Manhattan Beach, CA (US)

Correspondence Address: CONNOLLY BOVE LODGE & HUTZ LLP, P.O. BOX 2207, WILMINGTON, DE, 19899, US

(73) Assignee: MEDSOLVE TECHNOLOGIES, INC.

(21) Appl. No.: 11/618,682

(22) Filed: Dec. 29, 2006

(51) Int. Cl. A61M 1/00

(52) U.S. Cl. 604/67

(57) ABSTRACT

A system for infusing liquid to a body includes an infusion device, a network interface with a cell phone and wireless link and a network server system capable of communication with the infusion device through the network interface. The server system has access to a file of information specific to the controller for the infusion device. The infusion device includes a source of infusion fluid, a delivery port, a pump between the source of infusion fluid and the delivery port and a controller capable of programmable pump rate and sequence. A method for infusing liquid to a body includes controlling infusion using a programmable controller, establishing a file of information specific to the controller accessible to an extended area network server system and remotely transmitting commands to the controller. A sensor generates a diagnostic signal indicative of the magnitude of a constituent of liquid in the body and a routine provides an alarm signal when the magnitude exceeds a preset range.

### BACKGROUND OF THE INVENTION

The field of the present invention is infusion systems.

Today, diabetes is understood to be reaching epidemic proportions in the United States, it remains as a worldwide problem as well. Diabetes brings with it a host of medical problems which are aggravated by conventional medicament dosage schemes. Traditional shots and the like are intermittent which can induce a cyclical plethora and paucity of medicament, typically insulin. Recent efforts have been undertaken to eliminate such harmful cyclical variations through continuous administration of medicament. Additionally, devices and controls have been designed to provide both a basal rate for sustained infusion and periodic boluses to accommodate the ingestion of carbohydrates, in spite of these advantageous capabilities, difficulties remain in accommodating the correct infusion regimen, clinician control and payer's oversight.

A wide variety of prior systems having applicable components and processes exist. Reference is made to U.S. Pat. Nos. 4,898,578; 5,205,819; 5,630,710; 6,852,104; and 7,018,360. Reference is also made to U.S. Patent Publications Nos. 2002/0029776; 2003/0032867; 2003/0163088; 2004/0220551; and 2005/0137573. The disclosures of the foregoing patents and patent publications are incorporated herein by reference.

In addition to the infusion of insulin for diabetes, infusion is useful for other purposes in bodies of both humans and animals. The types of liquids that can be delivered include, but are not limited to, insulin, antibiotics, nutritional fluids, total parenteral nutrition or TPN, analgesics, morphine, hormones or hormonal drugs, gene therapy drugs, anticoagulants, analgesics, cardiovascular medications, AZT and chemotherapeutics. The types of medical conditions treatable by infusion include, but are not limited to, diabetes, cardiovascular disease, pain, chronic pain, cancer, AIDS, neurological diseases, Alzheimer's Disease, ALS, Hepatitis, Parkinson's Disease or spasticity.

In spite of the value of such systems to more consistently, uniformly and frequently supply and modulate medicament, issues remain. Failures can occur in equipment and in the body itself. Further, the advent of such equipment allows more at risk individuals to be away from the caregiver. Feedback of conditions can help to address such issues and can potentially provide freedom to even more at risk individuals.

### SUMMARY OF THE INVENTION

The present invention is directed to method and apparatus for infusing liquid to a body. The system contemplates the employment of an infusion device including a controller capable of programmable rate and time sequences. A separate communication device is employed in the infusion system as is a sensor monitoring the magnitude of a constituent of liquid in the body relevant to the infusion.

In a first separate aspect of the present invention, an infusion system includes the infusion device and a two-way communication device communicating with the controller through electrical contacts or a wireless link. One of the controller or the two-way communication device has a preset range limit for the magnitude of the constituent of liquid in the body. The two-way communication device has an alarm activated by the sensor when the measured constituent exceeds the limit.

In a second separate aspect of the present invention, a method of infusing liquid to a body includes regulating infusion to the body using the programmable controller. Infusion is controlled to the body using the controller responsive to the magnitude of the constituent. A preset range limit is established for the magnitude of the constituent and an alarm is activated when the range limit is exceeded.

Accordingly, it is an object of the present invention to provide improved method and apparatus for the infusing of liquid to a body. Other and further objects and advantages will appear hereinafter.

### BRIEF DESCRIPTION OF THE DRAWINGS

FIG. 1 is a cross-sectional side view of an infusion device.

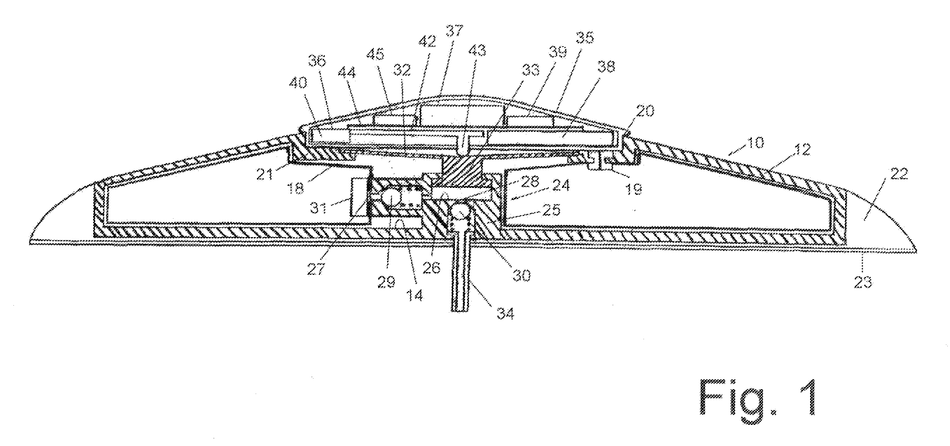

FIG. 2 is schematic view of a network system.

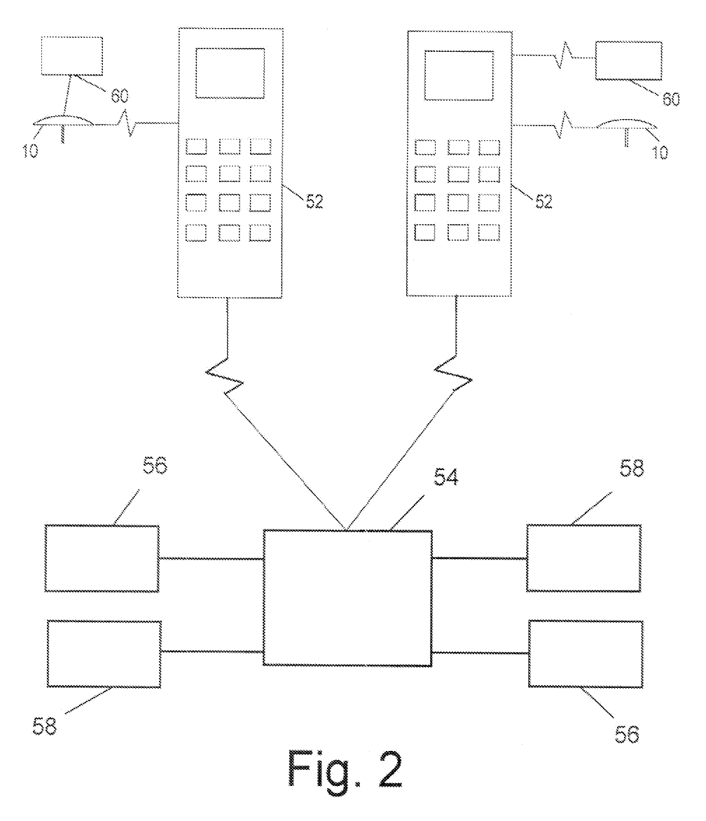

FIG. 3 is a logic diagram of the network system of FIG. 2 to serve a patient being infused.

FIG. 4 is a logic diagram of the network system of FIG. 2 for a connection routine between a two-way communication device and an infusion device.

FIG. 5 is a logic diagram of the network system of FIG. 2 for clinician input.

FIG. 6 is a logic diagram of the network system of FIG. 2 for a connection routine between the server system and the two-way communication device.

FIG. 7 is a logic diagram of the range limit for the magnitude of a constituent of liquid in the body.

### DETAILED DESCRIPTION OF THE PREFERRED EMBODIMENT

Turning in detail to a preferred embodiment of a system for infusing liquid to a body, the system presented in U.S. patent application Ser. No. 11/548,238, filed Oct. 10, 2006, is contemplated for employment with this invention and the disclosure of this application is incorporated herein by reference.

An infusion device, generally designated 10, includes a housing 12 conveniently circular in plan with a preferably low profile and a fiat base. Shapes other than circular are also possible and the base may have some concavity for conformance to a body shape. With a circular shape, the housing 12 most conveniently defines an annular space 14 for a reservoir to provide a source of infusion fluid. The reservoir may be defined by the annular space 14 or may include an internal annular bladder 18 of PTFE or nonplasticized PVC with an elastomeric fill port 19. A circular opening 20 is centrally located in the top of the housing 12 with a recessed mounting flange 21 as most convenient with the circular shape of the housing 12. The housing 12 further includes an elastomeric overmold periphery and mounting surface 22 for soft edges and comfort. The infusion device 10 is intended to be placed on the skin of the body to be infused and held in place by such means as an adhesive coating 23, tape or other medical retaining system.

A metering element is provided in the housing 12. In the preferred embodiment, the metering element is a pump, generally designated 24. Other possibilities for the metering element include a valve to vent pressurized liquid or a single stroke piston dispensing through a restricted orifice. A central boss 25 concentrically located within the housing 12 defines a pump body with a pump chamber 28, an inlet 27 and an outlet 28. The pump formed thereby is conveniently centrally located within the housing 12 in this preferred embodiment. The pump includes two one-way valves 29, 30 each defined by a ball biased against a seat by a coil spring. A filter 31, which passes liquid but blocks gas, is located at the inlet 27.

A circular diaphragm 32 is shown extending across the central opening 20 of the housing 12 attached to the circular recessed mounting flange 21. The diaphragm 32 is resilient, being of elastomeric material and includes a centrally positioned integral piston 33. The piston 33, operating as a movable pumping element, extends to the pump chamber 26 formed in the central boss 25 to vary the chamber volume. The diaphragm 32 is in radial tension to create a restoring force to maintain the piston biased toward one end of its stroke in the pump chamber 26.

The housing 12, with the bladder 18, the pump 24 and a delivery port to the body defined by a rigid cannula 34, is considered disposable and is fabricated in an inexpensive manner. The delivery port 34 to the body contemplates single use. The capacity of the reservoir is intended to exceed the demand for medicament during the full term of use which may be some multiple of a twenty four hour period depending on the expected volume of use.

The infusion device 10 further includes a second housing 35 which is not considered disposable at the same rate as the housing 12 and has multiuse capability with a plurality of disposable housings 12. The second housing 35 is placed in the circular opening 20 and can be secured by an interference fit, interlocking flanges or a threaded engagement to define an engagement. The second housing 35 includes a pump driver 36, a pump controller 37 and a battery 38.

The pump driver 38 includes a mounting block 40 that retains an actuator arm 42 which is a laminated strip with one spring leaf and a piezoelectric strip that deforms the arm 42 when voltage is applied. An actuator member 43 at the end of the arm 42 extends from the arm 42 through the housing 35 to operatively engage the piston 33 of the diaphragm 32. The driver 36 acts in one direction to force the piston 33 into the pump chamber 26 while the tension in the diaphragm 32 drives the return stroke. Other drives include a screw oscillating about its axis to advance the piston using a nanomotor or magnetic contacts to drive the oscillations.

The battery 38 may be a conventional watch battery. Alternatively, a rechargeable battery may be employed which can be recharged by an induction charger or by more conventional means. The battery 38 powers the driver 38 through the controller 37.

The controller 37 is electronic and is programmable for delivering a range of infusion rates and timing of the sequences of actuation with a capability to oscillate power delivery from the battery 38 to the pump driver 38. Through this programming, a basal rate can be controlled as well as periodic boluses. The controller 37 also can provide device ID and monitor such parameters as battery life. Alarm modes and volume, liming of communication and other infusion device functions can be added to the function of the controller 37. Being programmable, a microcontroller with memory is integrated on a circuit board 44. Other elements included on the board(s) 44 as needed or advantageous include a battery monitoring circuit, a power switch, a DC/DC converter, a voltage regulator, an LED and driver, an alarm generator, a jack or antenna, an induction coil and a crystal reference with the microcontroller.

The controller 37 includes a wireless link 39 for communication to and from the controller 37 using a radio chip on the circuit board 44 to transceive commands to the controller and unit identity and device status from the controller 37. The radio chip 39 uses a local area communication standard. Bluetooth®, NFC and Wibree™ communication standards can be used, with NFC requiring greater proximity of the components for communication.

A programmable multi-function device capable of two-way communication with the controller 37 and separate from the infusion device 10 is employed for communication to the wireless link 39 using the local area communication standard. This device, as a two-way communication device 52, is in turn in communication with an extended area network. In the preferred embodiment, the two-way communication device 52 is a cellular telephone preferably with Java-enabled program capability. A hand held computer with a wireless data link such as a BlackBerry® a Treo™ or other similar device, with the computer portion often identified as a “PDA”, is also contemplated. The phrase “two-way communication device” is intended to include all such possibilities while the terms “cellular phone” and its variations and the term “PDA” are intended to include those devices which access a public network for communication and data transfer. Such devices can advantageously download selected programs from the internet as may be used for glucose monitoring using a sensor or the like.

The extended area network with which the two-way communication device 52 is communicating may, therefore, be a cellular telephone network, a wide area network such as the internet or a private wide area network or a combination of technologies as systems become more integrated and the phrase “extended area network” is intended to include all such possibilities.

In a preferred embodiment, the protocol is through text communication, however, as for example, diabetics can have vision problems as a symptom of that disease, voice communication to the two-way communication device 52 from the extended area network may be preferred. Response through the extended area network by cell phone keypad or voice would also be possible.

A server system 54 compatible with the extended area network is in selective communication with the two-way communication device 52 through the extended area network. This system 54 has access to a file of information specific to the controller 37. When connected through the extended area network, the server system 54 can provide operating instructions to the two-way communication device 52 for programming the controller 37 for infusion rate and timing sequences. The server system 54 may be employed to communicate in real time with the controller 37 through the extended area network and the wireless link 39 by relaying commands through the two-way communication device but more facilely can download programming and data to the programmable two-way communication device 52 for later and/or repeated communication with the controller 37.

The file of information specific to the controller accessible by the server system is accessible for creating and amending instructions from a control terminal 56. The control terminal is accessible by a clinician for inputting and changing the file through network access.

A monitoring terminal 58 can also be employed. The monitoring terminal 58 has limited access to the file of information specific to the controller. The terminal is intended to be employed by payers to service providers, e.g., medical insurance companies. The payer is anticipated to be interested in at least the incidents of inputting and changing of the file of information specific to the controller by the clinician. Additionally, the payer may be set up to see the results of clinician control for purposes of evaluating the quality of service. In the case of diabetes, this may include the stability of a blood glucose level in the body of the infused person.

Blood glucose levels or other controlled constituents of liquid in the body may be measured and input to the sewer system 54. The monitoring can be undertaken by a sensor 60 automatically generating a diagnostic signal indicative of the magnitude of a detected substance relevant to the infusion, such as blood glucose. A radio chip and battery similar to that employed for the infusion device 10 may be employed in association with the sensor 60. Alternatively, the sensor 60 may communicate through the wireless link radio chip 39 of the controller 37 and even share the same energy source 38. The diagnostic may be employed by the controller 37 directly, such as through a table downloaded as commands to the controller 37. A variable of the infusion device would be amended in accordance with the look-up table responsive to a magnitude of the diagnostic input. Alternatively, the diagnostic may be transmitted to the two-way communication device, either directly or through the controller 37, where a downloaded program from the server system employs the diagnostic to generate commands to the controller 37. Such sensors 80 are available from the insulin infusion industry. Examples are disclosed in U.S. Pat. Nos. 5,741,211 and 6,892,085, the disclosures of which are incorporated herein by reference.

A preset range limit for the magnitude of the constituent of liquid in the body relevant to the infusion may be programmed into either the controller 37 or into the two-way communication device 52. The range of the limits, both over and under the target desired level, is determined by the caregiver and input through the server system 54. The diagnostic signal from the sensor is monitored by either the controller 37 or into the two-way communication device 52. The signal may also go to either. Further, even if the signal goes to the controller 37, the signal may be processed or passed along to the two-way communication device 52. One of the processing units 37, 52 includes a routine to check the magnitude of the constituent as represented by the diagnostic signal. When the preset limit is exceeded, either over or under, the processing unit generates a signal activating an alarm signal in the two-way communication device 52.

In addition to the foregoing, the same infusion system can serve multiple patients, each with an infusion device 10 and a two-way communication device 52. In this case, the server system 54 includes access to a multiple number of files of operating instructions. With a full medical service, many patients each wearing an infusion device 10 and communicating through a two-way communication device 52, multiple control terminals accesses by multiple clinicians and multiple monitoring terminals 58 serving multiple payers can function from the same server system 54. Safeguards are provided to insure the electronic communication appropriately addresses the correct infusion device and correct file of operating instructions. Dual identification handshakes, passwords and the like are programmed into the system.

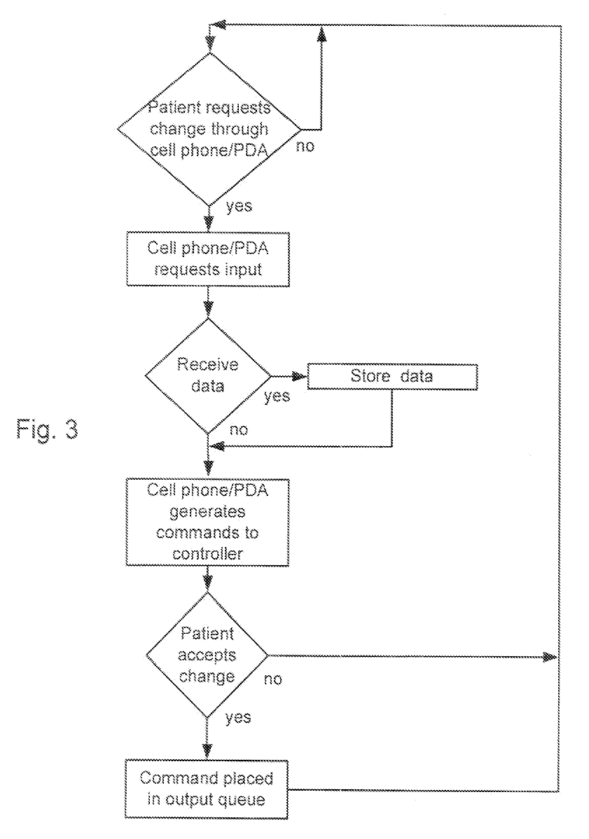

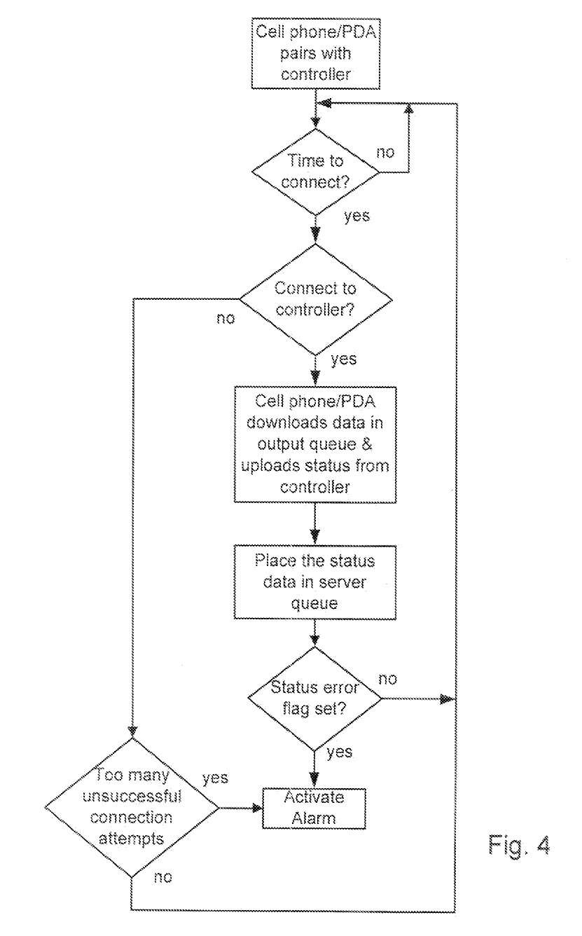

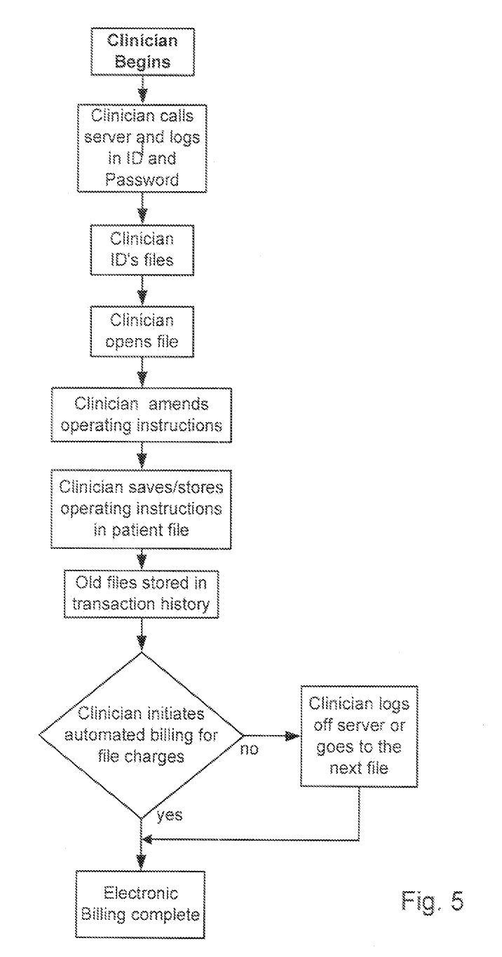

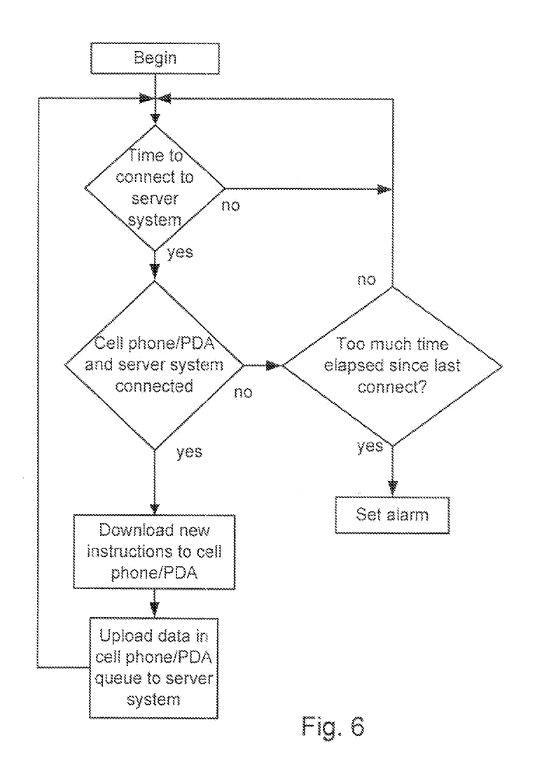

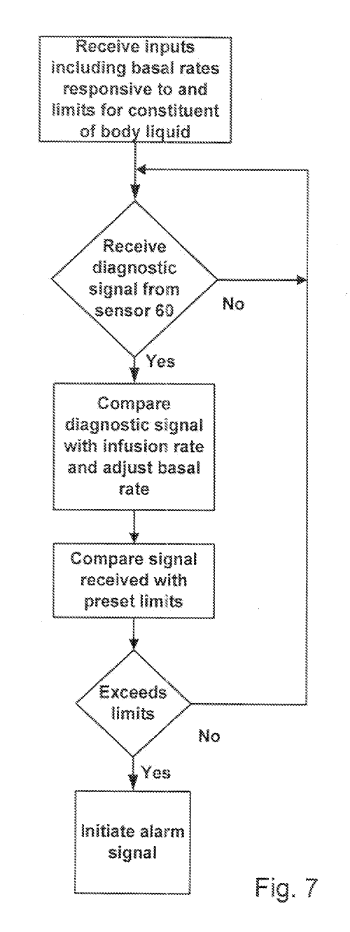

Turning to system operation, a method for infusing liquid into a body is also presented. FIG. 3 illustrates communication logic with the patient interacting with the two-way communication device 52. FIG. 4 illustrate the logic steps for the two-way communication device 52 communicating with the controller 37. FIG. 5 illustrates the logic steps for the control terminal interfacing with the server system. FIG. 6 illustrates the logic steps for the server system 54 communicating with the two-way communication device 52. FIG. 6 illustrates the logic steps for initiating a warning signal. FIG. 7 illustrates the logic steps for initiating an alarm when the diagnostic signal from the sensor 60 indicates the exceeding of a preset range of limits.

In brief and looking to the Figures, the process of the patient interacting with the two-way communication device 52 begins with the patient inputting a request to the two-way communication device 52. In response, the two-way communication device 52 requests appropriate input. The input requested may be specifically responsive to the patient request or may follow certain repeated routines or both. The requested input may include blood glucose levels, anticipated carbohydrate intake, changes to settings, and the like. If the data is received, it is stored in the two-way communication device 52. In either event, the two-way communication device 52 generates appropriate commands as dictated by the program from the patient file, which commands are responsive to the patient request. The patient then accepts or rejects the change and, if accepted, the change is placed in the output queue. If rejected, the process is repeated.

The routine for the two-way communication device 52 to connect with a paired controller 37 is shown to include timing between the two. When the timing is correct, an attempt is made by the two-way communication device 52 with the controller 37 energized by synchronized timing to make contact, if the connection is made, the two-way communication device 52 downloads data from the output queue to the controller 37 and uploads status from the controller 37. If the status does not include an error flag, the process is recycled. If there are too many unsuccessful attempts to connect with the controller 37 or the status error flag is set, an alarm is activated. With a status error flag set in the controller 37, an alarm may be activated at the controller at that time. An alarm associated with the two-way communication device 52 and/or the server system 54 would be activated upon recognizing the status error flag or counting too many unsuccessful connection attempts.

The logic steps for use of the control terminal 56 is initiated by the clinician logging in. The appropriate file is retrieved and operating instructions are amended if the stored history, changes or new prescriptions are in place. The amendments are made and stored along with historical data. The clinician may further initiate automated billing for file charges and the process is complete.

For the communication between the server system 54 communicating with the two-way communication device 52, a time or incident initiation from either the two-way communication device 52 or the server system 54 initiates a connection. When connected, new and amended instructions are downloaded from the server system 54 to the two-way communication device 52 and data in the queue of the two-way communication device 52 is uploaded to the server system 54. When too much time is elapsed since the last connection, an alarm is set.

The system uses input of diagnostic signals from the sensor 60 for both adjustments to the basal rate, either automatic or through interaction with the patient, and for initiating an alarm when a preset range of limits is exceeded as illustrated in FIG. 7. Variously, the controller 37 and/or the two-way communication device 52 is programmed with a relationship of basal rate to diagnostic magnitude input from the sensor 60. Further, a preset range of limits for the diagnostic magnitude input is programmed as well. Sensing and transmission of results from the sensor 60 then takes place on either an automatic timed basis or upon inquiry from either the controller 37 or the two-way communication device 52. The signal received is then employed to set a basal rate in accordance with preprogrammed instructions. The signal received is also compared with the preset limits to determine if the limits are exceeded, by either too much or too little of the measured constituent in the body liquid, if the range of limits is exceeded, a signal is generated to set off an alarm in the two-way communication device 52

The programmable controller 37 is employed to control infusion into the body. To undertake that control, a file of information specific to the controller 37 is established and accessible to the server system 54. The server system 54 is associated with the extended area network preferably national or international in scope such as employs internet or cell phone technology. The server system 54 preferably has access to a great many files of information specific to controllers 37 to serve a large number of infusion patients. All such patients may be served over the extended area network, to their individual two-way communication devices 52. From the server system 54, operating instructions including programming are transmitted over the extended area network to two-way communication devices 52. In the preferred embodiment above, the two-way communication devices 52 are Java-enabled cell phones with the extended area network being a cellular or satellite telephone network.

The creation or modification of information specific to a patient infusion device 10 is accomplished through the control terminal 56 by amending the updatable file of operating instructions. This function is illustrated in the logic diagram of FIG. 5. The control terminal is typically operated by a clinician remote from the server system. The rules of control for the controller 37 are established within the file of operating instructions. Such rules, as determined by the clinician, may include a set basal rate or a range of permitted basal rates. Such settings may be arranged on the basis of periods of the day to track common changes in infusion needs as more specifically fine tuned for each individual. The file further contains rules for bolus administration. Limits and specific values may be incorporated into the file of information specific to the controller for the specific patient, which may include rules responsive to estimated carbohydrate intake. The file can also keep track of the history of activity by the clinician for billing purposes. The file of updatable information also receives input from the two-way communication device 52, which is principally historical or indicative of device status.

With the use of a programmable two-way communication device 52, the server system 54 may download programming including operating instructions at regular or requested intervals to the two-way communication device 52. This function is illustrated in the logic diagram of FIG. 3. Such instructions may be principally programming specific to the patient as dictated by the clinician. The programming in the two-way communication device 52 is appropriately updated at a frequency which maintains adequate currency from the server system 54. The routine may include having the patient query the server system 54 each time the patient makes a request. The patient, or caregiver with the patient, may repeatedly interact with the two-way communication device 52 to request basal rate changes and boluses between changes in the basic program input from the server system 54 to the two-way communication device 52. Such independent operation is of particular benefit when network access to reach the server system 54 is unavailable.

The patient input includes secure identification and may be undertaken using keypad input or voice communication to the programmable cell phone 52 or to the other devices discussed above. The two-way communication device 52 can then prompt orally or visually, for the necessary input. Such input appropriately would include the estimated amount of carbohydrates in the case of diabetics which have just been ingested or are to be ingested for a bolus. Periodically current blood glucose level can be demanded of or voluntarily submitted by the patient to be entered automatically on a periodic basis from a sensor 60. These functions are seen in the logic diagram of FIG. 3. The degree of control afforded the patient is also determined in the programming by the clinician.

The requested basal rate changes and bolus requests input to the two-way communication device 52 are transmitted as program commands. The commands are sent through the wireless link 39 to the programmable controller 37. The commands for implementation are maintained within the boundaries of discretion dictated by the operating instructions to the two-way communication device 52 from the patient file accessible by the server system 54.

Currency is also maintained in the patient file accessible to the server system 54 by return from the two-way communication device 52 of data regarding the history of requests, rate changes, boluses administered, recorded changes in constituent fluid status, system and component status and other possible input such as demographics or diabetes centers. Such information is useful to the clinician for subsequent treatment and to any analysis of treatment efficacy. This input from the two-way communication device 52 may follow some period of operation independent of the server system 54 or be timed to more closely monitor patients in unstable circumstances. The server system 54 can also generate automatic alerts to clinicians when malfunctions are sensed or boundary values are exceeded as discussed below. The system is designed to check periodically as well as be connected when requests or changes are made.

The monitoring of care of a patient and efficacy of the treatment can be undertaken from analysis of the patient file from any authorized terminal. This function is illustrated in the logic diagram of FIG. 8. The monitoring terminal 58 is provided with network access. Such access may be limited to information retrieval and may further be limited to specific information. Use of the monitoring terminal 58 may be employed for generating payments to providers for accumulating activity of the clinician such as monitoring constituents of liquid in the body, amending basal rate and bolus authorizations and the like. The transmission of information prompting billing may occur through generation of a communication by the clinician or by monitoring by the payer. The payer may then generate payments to the service providers consistent with such activity. Additionally, the monitoring function through the monitoring terminal 58 may include oversight of the monitoring effectiveness and quality of control over the controlled constituents of liquid in the patient. Through such oversight functions, risk factors can be accessed. The inability to control glucose levels in a diabetic patient, for example, would signal an increased level of risk of complications.

Communication between the controller and the two-way communication device is arranged to conserve battery power in the infusion device. Rather than have the wireless link prepared to receive communication from the two-way communication device at any time, the two-way communication device and controller sync clocks each time they do communicate. Further, a time interval, say five minutes, is set when the controller is to turn on the wireless link and the two-way communication device sends a signal to the controller. The signal may be the initiation of a change in infusion rate and/or sequence or communication of infusion device status and a further sync of the clocks for another period, thereby setting up scheduled data shuttles. The logic steps for this operation are illustrated in FIG. 4.

When the user requests a change or the server downloads a change to the two-way communication device under the battery conservation scheme, the two-way communication device is scheduled for a download to the controller. At the appointed interval, the controller switches on and the two-way communication device transmits the change to the controller. Where control is time critical a short time interval can be used for the scheduled data shuttles.

The schedule may be used to monitor and signal concern, if the communication device fails to establish a connection to the controller through the wireless link after a preprogrammed number of tries, the two-way communication device will alert the user through an audio or vibrational alarm as the infusion device has either malfunctioned, including having a dead battery, or the device is out of range of the two-way communication device.

A further failsafe mechanism can be implemented by requiring the two-way communication device to signal the server on a regular basis that communication to the infusion device is maintained and that an error-free status byte has been received. If the server does not receive this information, either the infusion device has failed or is out of range of the two-way communication device or the two-way communication device has failed or is out of range of any transfer station. The server can then send an alert such as by calling an emergency number.

Thus, an improved method and apparatus for the infusion of liquid to a body is disclosed. While embodiments and applications of this invention have been shown and described, it would be apparent to those skilled in the art that many more modifications are possible without departing from the inventive concepts herein. The invention, therefore is not to be restricted except in the spirit of the appended claims.

What is claimed is:

1. A system for infusing liquid to a body, comprising
an infusion device including a source of infusion fluid, a delivery port to the body, a metering element coupled between the source of infusion fluid and the delivery port and a controller capable of programmable infusion rate and time sequences;
a two-way communication device capable of communicating with the controller and including an alarm signal;
a sensor capable of generating a diagnostic signal indicative of the magnitude of a constituent of liquid in the body relevant to the infusion and being in communication with one of the controller or the two-way communication device, one of the controller or the two-way communication device having a preset range limit for the magnitude of the constituent of liquid in the body and a routine for activating the alarm signal in the two-way communication device when the preset range limit is exceeded.

2. The system for infusing liquid to a body of claim 1 further comprising
a server system capable of communication with the two-way communication device through an extended area network and having access to a file of updatable information specific to the controller.

3. The system for infusing liquid to a body of claim 2, the file including the preset range limit for the magnitude of the constituent of liquid in the body, the server system being capable of downloading the preset range limit to the two-way communication device.

4. The system for infusing liquid to a body of claim 3, the sensor being in direct communication with the controller.

5. The system for infusing liquid to a body of claim 4, the two-way communication device downloading the routine to the controller to signal the two-way communication device to activate the alarm signal when the magnitude of the constituent of liquid in the body is outside the preset range limit.

6. The system for infusing liquid to a body of claim 4, the controller transmitting the diagnostic signal from the sensor to the two-way communication device, the two-way communication device including the routine to activate the alarm signal when the magnitude of the constituent of liquid in the body is outside the preset range limit.

7. The system for infusing liquid to a body of claim 3, the sensor being in direct communication with the two-way communication device.

8. The system for infusing liquid to a body of claim 7, the two-way communication device including the routine to activate the alarm signal when the magnitude of the constituent of liquid in the body is outside the preset range limit.

9. A method for infusing liquid to a body, comprising the steps of
communicating between a programmable controller and a two-way communication device including programming the programmable controller using the two-way communication device and monitoring the programmable controller;
sensing the magnitude of a constituent of liquid in the body relevant to the infusion;
transmitting data from the sensor to one or both of the programmable controller or the two-way communication device;
controlling infusion to the body using the programmable controller responsive to the magnitude of the constituent of liquid in the body;
establishing a preset range limit for the magnitude of the constituent of liquid in the body in one of the programmable controller or the two-way communication device;
activating an alarm signal in the two-way communication device when the preset range limit is exceeded.

10. The method for infusing liquid to a body of claim 9, the step of transmitting being to the programmable controller, the step of programming including establishing a routine in the programmable controller to monitor the data, to compare the data with the preset range limit and to send an alarm activation signal to the two-way communication device when the preset range limit is exceeded using the step of communicating.

11. The method for infusing liquid to a body of claim 9, the step of transmitting being to the programmable controller, the step of communicating including sending the data to the two-way communication device, the step of monitoring including to monitor the data received from the programmable controller, to compare the data with the preset range limit and to generate an alarm activation signal to initiate the step of activating.

12. The method for infusing liquid to a body of claim 9, the step of transmitting being to the two-way communication device, the step of monitoring including to monitor the data received, to compare the data with the preset range limit and to generate an alarm activation signal to initiate the step of activating.
